(19) United States
(12) Patent Application Publication
(10) Pub. No.: US 2023/0218754 A1
(43) Pub. Date: Jul. 13, 2023

(54) PHARMACEUTICAL COMPOSITIONS WITH ANTI-RANKL ANTIBODIES, CALCIUM AND VITAMIN D

(71) Applicant: CHRISTOPH KARL, TAEGERWILEN (CH)

(72) Inventors: CHRISTOPH KARL, TAEGERWILEN (CH); RUEDIGER THIEROLF, BABENHAUSEN (DE)

(73) Assignee: CHRISTOPH KARL, TAEGERWILEN (CH)

(21) Appl. No.: 18/167,097

(22) Filed: Feb. 10, 2023

Related U.S. Application Data
(63) Continuation of application No. 16/301,737, filed on Nov. 15, 2018, now abandoned.

(30) Foreign Application Priority Data
May 20, 2016 (DE) 10 2016 006 557.5

(51) Int. Cl. A61K 39/395; A61K 33/06; A61P 19/10; A61K 31/592; A61K 31/593; C07K 16/28; A61P 35/00; A61K 39/00

(52) U.S. Cl. CPC A61K 39/3955; A61K 39/395; A61K 33/06; A61P 19/10; A61K 31/592; A61K 31/593; C07K 16/2875; A61P 35/00; C07K 2317/56; A61K 2039/545; A61K 2039/54; C07K 2317/76; C07K 2317/622; A61K 2039/505

(57) ABSTRACT

A method of administering a pharmaceutical composition which includes anti-RANKL antibodies, calcium and vitamin D to a patient in need thereof. The method includes subcutaneously administering the anti-RANKL antibodies at a dose of 60-180 mg in 1-3 ml of solution every four weeks, orally administering the calcium at a dose of 400 mg daily, and orally administering the vitamin D at a dose of 800-1200 IU daily. The patient in need thereof has skeletal-related complications due to solid tumors, and the administering of the pharmaceutical composition comprising anti-RANKL antibodies, calcium and vitamin D to the patient in need thereof at least one of treats and prevents hypocalcemia induced by anti-RANKL antibody therapy. The calcium and the vitamin D are provided as a combined single daily dose in a form of effervescent granules, swallowable tablets, swallowable capsules, chewable tablets, or ready-to-use granules.

### CROSS REFERENCE TO PRIOR APPLICATIONS

This application is a continuation of application Ser. No. 16/301,737, filed on Nov. 15, 2018, which is a U.S. National Phase application under 35 U.S.C. § 371 of International Application No. PCT/EP2017/062087, filed on May 19, 2017 and which claims benefit to German Patent Application No. 10 2016 006 557.5, filed on May 20, 2016. The International Application was published in German on Nov. 23, 2017 as WO 2017/198811 A1 under PCT Article 21(2).

### FIELD

The present invention relates to pharmaceutical compositions comprising anti-RANKL antibodies, calcium, and vitamin D that are suitable for the treatment and/or prevention of disorders of bone metabolism and therapy-induced adverse effects such as hypocalcemia caused by the action of the anti-RANKL antibodies.

### BACKGROUND

The treatment of disorders of bone metabolism, such as osteoporosis, tumor diseases including induced bone loss (CTIBL, cancer treatment-induced bone loss), Paget's disease, or hypocalcemia, constitutes an important focus of efforts in medical research. Human bone is composed of an organic matrix that is combined with inorganic salts—primarily calcium and phosphate in the form of hydroxyapatite (Ca₃(PO₄)₂.Ca(OH)₂). As an underlying principle, bone physiology in humans is based on constant formation and resorption of bone tissue (bone turnover). Bone formation and resorption in the ongoing remodeling process are regulated hormonally (essentially by estrogen and parathyroid hormone (PTH)) and by signal transduction pathways (essentially RANKL (receptor activator of nuclear factor-kB ligand)/OPG (osteoprotegerin) and Ca/PTH/vitamin D3). While more bone is formed than resorbed in childhood, and especially in puberty, with increasing age there is an increasing tendency toward “net” bone resorption, particularly in women during and after menopause.

Throughout the course of human life, bone is constantly exposed to physiological and mechanical stresses, and adapts accordingly. This was first described in 1862 by Julius Wolff, and Wolff's law constitutes the cornerstone of biomechanical understanding of bone. Wolff was able to show in a study of the femoral head that the form of bone adapts to its function, and that bone degenerates unless it is constantly subjected to a load. In the course of “remodeling,” therefore, bone not subjected to a load is resorbed, a phenomenon that was examined in bed rest studies of patients with absolute bed rest for a period of 60 days. Bone subjected to a strong mechanical load, on the other hand, is strengthened along the lines of force in question, which was demonstrated using the example of the forearm bones of tennis players.

On application of a force to the bone, in addition to regular remodeling processes, microscopic damage or interruptions in continuity (fractures) occur with and without defect formation, and these can heal without scarring. In the case of inorganic materials, repeated loading below the breaking point leads to material fatigue/material fatigue fracture, in which small cracks develop and grow until they reach a critical size and the material breaks. As a semi-brittle material, bone can avoid fracturing by absorbing energy by forming tiny cracks (so-called micro-cracks). Micro-cracks were described as early as 1960 by Harold Frost and are sharply delineated cracks 50-100 μm in length that occur primarily in interstitial bone. As a rule, in accordance with the biomechanical load in question, their longitudinal axis is longer than their transverse axis, they occur in the case of physiological repetitive loading such as walking or running in the trabecular and cortical bone, and they occur to a significantly increasing degree with increasing age.

As a rule, micro-cracks remain clinically unnoticed because they are continuously repaired and healed in the course of remodeling. The cracking results in apoptosis of osteocytes that release factors such as RANKL. These initiate osteoclastic resorption, resulting in osteoblastic bone regeneration.

While healthy bone inhibits and repairs the progression of microscopic damage, in aged bone or e.g., under suppression of remodeling by highly potent antiresorptive agents such as e.g., bisphosphonates or anti-RANKL antibodies, accumulation and in some cases increased fracture susceptibility may occur, e.g., in atypical femoral fractures.

Osteoblasts, osteoclasts, and osteocytes are primarily involved in the ongoing bone regeneration processes taking place in bone remodeling. While osteoclasts break down the bone, osteoblasts can regenerate it.

Finally, the role of the osteocytes has not yet been clarified in detail. These are terminally differentiated cells of osteoblastic origin embedded in the bone that are connected to and communicate with one another. In the event of injury to osteocyte branches, e.g., in the case of the above-described micro-cracks, osteoclastic resorption is first initiated, and this is then again followed by osteoblastic bone formation. The interaction between osteoblasts, osteoclasts, and osteocytes is essentially controlled by the RANKL/OPG signal transduction pathway (differentiation of osteoclasts) and the WNT/DKK/SOST pathway (WNT is a signal protein composed of wingless (Wg) protein and int-1 protein; DKK=Dickkopf; SOST=symbol for the protein sclerostin)) (differentiation of osteoblasts).

The primary pathways involved in bone formation are the WNT pathway for differentiation of osteoblasts and the PTH signal transduction pathway. The differentiation of hematopoietic stem cells to osteoclasts is controlled via the RANKL/OPG signal transduction pathway.

Bone serves as a calcium storehouse for the human body and is thus decisively involved in calcium phosphate homeostasis. By means of bone formation and resorption, excess calcium reserves can be stored from the blood or again be made available as needed.

In a wide variety of disorders of bone metabolism, this sensitive balance is disturbed. The most frequent disorder of bone metabolism is osteoporosis. Other disorders that affect bone metabolism include Paget's disease, osteogenesis imperfecta, primary bone tumors, as well as multiple myeloma (plasmacytoma), osteosarcoma, and bone metastases. Complications caused by bone metastases/bone tumors are referred to as “skeletal related events” (SREs). The term “skeletal related events” (SREs) includes acute events such as pathological bone fractures, spinal cord compression, bone pain, or tumor-induced hypercalcemia and therapeutic interventions such as bone irradiation or bone surgery, which may be carried out for example in patients with bone metastases or primary bone tumors.

Osteoporosis causes a reduction in bone density, which together with impaired bone quality leads to an increased fracture risk. The WHO currently defines osteoporosis as a measurable reduction in bone density of less than −2.5 standard deviations (so-called T score) in a health collective (women 30 years of age) as measured by DXA (dual-energy X-ray absorptiometry). When a bone fracture has occurred, one speaks of manifest osteoporosis. Osteoporosis is thus a systemic disorder of the bone that frequently manifests as a fracture resulting from inadequate trauma. In Germany, approximately 6 million people suffer from the widespread disease osteoporosis, and osteoporosis patients suffer more than 720,000 fractures annually. Postmenopausal osteoporosis is by far the most frequent cause of vertebral and non-vertebral fragility fractures. The risk of a fracture increases considerably with increasing age.

In the first 5 years after menopause, with the discontinuation of estrogen production, one observes bone mass resorption that varies in severity among individuals. Postmenopausal bone mass loss can be as much as 15% per year and leads very rapidly in women with a low peak bone mass to the first osteoporotic fracture.

Deficient calcium supply and depletion of blood vitamin D levels lead on balance to calcium resorption by the bone. It is also to be expected that further pathologic mechanisms will occur with increasing age that directly or indirectly promote bone resorption. By age 65, for example, the capacity to absorb calcium ions from food has decreased by approximately 50% (compared to young people). Age-related comorbidities such as diabetes mellitus, impaired kidney function, immobility, or treatment with glitazones are only a few examples from the long list of risk factors that have been shown to have an additive effect on bone resorption and thus to promote a latent progression of osteoporosis.

FIG. 1 clearly shows that only approximately a third of the amount of calcium available in the digestive tract is resorbed. 65-70% of dietary calcium is excreted in the stool. The resorption process itself can additionally be positively or negatively affected by various factors. For example, food containing high concentrations of phosphates can inhibit calcium uptake to an extreme degree.

The primary hormones that control calcium homeostasis, in addition to the sex hormones (estrogen, testosterone), include calcitonin, parathyroid hormone (parathyrin) and calcitriol (the active form of vitamin D3). Calcitriol is essential for allowing absorption of calcium and phosphate in the small intestine. Moreover, calcitriol increases the reabsorption of calcium via the kidneys and stimulates bone mineralization (incorporation of calcium into the bone matrix).

A negative calcium balance occurs, for example, when the body excretes more calcium than it can reabsorb via the intestine. The result is increased bone resorption and secondary hyperparathyroidism if this state persists over a long period of time. In order to mobilize the “lacking” calcium from the bone, parathyroid hormone is formed by the parathyroid glands. As shown in FIG. 2, parathyroid hormone stimulates bone resorption and thus causes an increase in the blood calcium concentration. The calcium concentration therefore remains relatively constant in the blood at the expense of the bone, which releases its calcium in the case of a calcium deficiency in order to increase the level of calcium in the blood.

FIG. 3 shows a diagram of calcium losses with age. As early as the age of 40, insidious bone density loss of 2-3% per year begins in men and women. In more than 20% of women, there can be enormous increases in annual losses after menopause. The women affected suffer an often unnoticed loss of more than a quarter of their bone mass (=calcium) over a relatively short period of time and therefore have an extremely high risk of fractures in the further course of their lives. The disease “osteoporosis” is diagnosed too late in many cases—usually not until an osteoporotic fracture (i.e., a fracture without a traumatic event), for example, of the vertebrae, hip, or wrist, has already occurred.

An additional problem in affected men and women older than 50 years of age that is not to be underestimated is the increasing calcium gap resulting from a progressive negative calcium balance. As shown in FIG. 4, with increasing age, there is an increasing discrepancy between the daily calcium requirement and the real intake of calcium from the gastrointestinal tract. The age-related high requirement for calcium is essentially influenced by the following factors: decreasing calcium resorption in the gastrointestinal tract, calcium-poor diet (few dairy products), vitamin D deficiency, lack of exercise, required replenishment of the bone calcium storehouse (formation of new bone mass), increased parathyroid hormone in the blood (increased bone resorption and calcium losses), or impaired kidney function (reduced calcium reabsorption and less active vitamin D).

The human body only Ultimately has two possibilities in order to compensate for natural calcium losses via the skin and kidneys and in order to maintain the constant level of calcium in the blood necessary for life:

1) Mobilize calcium from the “bone calcium storehouse”. Drawback: The resorption of bone tissue necessary for this purpose can lead in the long term to losses of stability and fractures.

2) Sufficient resorption of calcium from the gastrointestinal tract and reabsorption of calcium in the kidneys. Both of these processes are controlled by vitamin D.

The biologically active form of vitamin D3 (1α,25-dihydroxycholecalciferol; calcitriol) promotes the absorption of calcium from the gastrointestinal tract into the blood. The maximum serum concentration is reached within three to six seconds after intake. In total, vitamin D promotes the intake of calcium into the body. An overdose can lead to vitamin D poisoning, as the body stores vitamin D. Vitamin D poisoning can result in severe demineralization of the bone, which in turn results in fractures. High calcium serum concentrations can at the same time lead to abnormal calcification of a wide variety of soft tissues. Kidney stones may also occur because of the increased renal calcium excretion.

Because of reduced resorption from the intestine, a vitamin D deficiency causes less dietary calcium to reach the blood.

A calcium and/or vitamin D deficiency is treated by supplementation with calcium and vitamin D-containing preparations. However, the use of calcium and vitamin D is not uncontroversial. The tolerability of calcium is being debated throughout the world, and some scientists consider calcium supplementation to constitute a cardiovascular risk. It is claimed that calcium increases the risk of cardiovascular events such as heart attack. The result of this debate in Germany has been that calcium supplementation is often not used.

In osteoporosis, calcium deficiency means aggravation of the pathology with an increased fracture risk.

In manifest osteoporosis, the bone mineral content is reduced (T score: <−2.5), and fractures have already occurred, for example, 1 to 3 vertebral fractures.

Long-term glucocorticoid therapy can lead to severe glucocorticoid or corticoid-induced osteoporosis with fractures. In administration of >5 mg of prednisone equivalent per day over a period of >3 months, preventive or therapeutic measures are recommended, particularly during the first six months after beginning therapy, in the event of major dose increases during the course thereof, and in the case of high-dose long-term therapy, one can expect a significant decrease in bone density, which should be taken into account in treatment. Glucocorticoid-induced bone loss has multiple causes. At the beginning of therapy, glucocorticoids cause an increase in bone resorption. Steroids inhibit the proliferation and function of osteoblasts and increase apoptosis thereof. They cause a decrease in bone neoformation in this manner. At the same time, they lead to a negative calcium balance by inhibiting intestinal calcium resorption and increasing urinary calcium excretion.

A further disorder of bone metabolism is Paget's disease, also known as osteodystrophy deformans, osteitis deformans, Paget's syndrome, or Paget disease, and in this disorder, the cause of which remains unknown, possible etiologies under discussion are genetic, viral, and environmental influences. Paget's disease is a chronic disorder of bone metabolism characterized by locally elevated bone remodeling processes with the risk of deformations, chronic pain, and fractures and articular, neurologic, and cardiologic complications.

Solid primary tumors, such as e.g., breast, prostate, lung, bowel, or bone cancer (e.g., osteosarcoma), can be manifested in the bone and thus cause bone metastases or bone disorders. Bone metastases are migrations of other tumors into the bones where they can cause pain and fractures. This is attributable to increased bone resorption or excessive production of inferior-quality bone tissue. This reduces the stability of the bones. Tumor-induced bone complications are characterized by a high morbidity rate, an increased fracture rate, nerve entrapment, and in some cases unbearable pain. If increased amounts of calcium are released from the bone and transferred into the bloodstream, tumor-induced hypercalcemia can develop. For example, the number of patients with moderate or severe bone pain (>4 points on the BPI-SF [brief pain inventory, short form]) with metastasizing breast, prostate, lung, bowel, or bone cancer (e.g., osteosarcoma) is greatest at over 80%, followed by neuralgia at approximately 10%, thus making this by far the most common symptom (Cleeland, C. S. et al., Ann. Onc. 2005, 16: 972-980).

The term “multiple myeloma” or “plasmacytoma” or “Kahler's disease” refers to a cancer of the bone marrow in which the antibody-producing cells (plasma cells) are sharply increased. These malignant plasma cells reproduce in an uncontrolled manner and form nonfunctional antibodies or parts thereof. The course of the disease can vary considerably, with moderate to highly malignant courses, which if left untreated are rapidly fatal. The symptoms are caused by growth of the cells or by the antibodies produced or fragments thereof, and can manifest themselves as bone pain, loss of bone mass, and fractures, accompanied by increased release of calcium into the blood, which can lead to tumor-induced hypercalcemia. The number of leukocytes decreases, while the large number of antibodies are deposited in the tissue and cause functional disorders of numerous organs, renal failure, and circulatory impairment.

Drugs for the treatment of disorders of bone metabolism are known in the prior art. In pharmaceutical treatment of osteoporosis, the primary objective is to stop pathological bone mass loss. The negative balance (disequilibrium between the formation and resorption of bone tissue) can be compensated for either antiresorptively by inhibition of osteoclasts (bone resorption) or anabolically by stimulation of osteoblasts (bone formation). Only when balance has been restored between the processes of formation and resorption and a sufficient supply of calcium and vitamin D is ensured can new bone mass be formed and the risk of osteoporotic fractures be reduced.

In order to treat osteoporosis, one can inhibit osteoclastic bone resorption by means of anti-RANKL antibodies and stimulate bone formation using anabolic drugs. The recombinant 1-34 fragment of parathyroid hormone is approved for the treatment of osteoporosis for this purpose.

Antiresorptive agents such as anti-RANKL antibodies are currently the standard for the treatment of metabolic bone disorders, and they require effective inhibition of bone resorption. This includes the use of this class of substances for the treatment of osteoporosis and—at sharply higher doses and shorter treatment intervals—for the treatment and/or prevention of tumor-induced skeletal complications.

Anti-RANKL antibodies or derivatives thereof are ordinarily used to treat the above-mentioned disorders such as osteoporosis or tumor-induced bone disorders.

Calcium remains in the bone as a result of the use of anti-RANKL antibodies. This causes inhibition of bone resorption to varying degrees, e.g., by parathyroid hormone. This poses the risk of hypocalcemia. Hypocalcemia is present when total serum calcium is below 2.2 mmol/1 (9 mg/dl). In pharmaceutical inhibition of osteoclasts, the calcium concentration in the blood can only be balanced by an external supply of calcium (orally or intravenously). This is shown in FIG. 5. PTH activates the release of calcium from the bone, but this is inhibited by the inhibition of osteoclasts due to the use of the anti-RANKL antibodies. An external calcium supply is therefore essential.

The risk of hypocalcemia is also significantly increased in the presence of secondary hyperparathyroidism accompanied by a vitamin D deficiency. In this commonly-occurring state of deficient vitamin D, increased amounts of parathyroid hormone are secreted in order to balance the serum calcium concentration. Parathyroid hormone indirectly increases the plasma calcium concentration by activating the osteoclasts.

Antiresorptive therapy aggravates this hypocalcemia because the osteoclasts are inhibited.

In oncological use of antiresorptive agents such as anti-RANKL antibodies in particular, an insufficient supply of calcium and/or vitamin D can be dramatically exacerbated. Because of their high antiresorptive potency, a so-called “red hand letter” was issued for both PROLIA (denosumab 60 mg) and XGEVA (denosumab 120 mg) in September of 2014, expressly instructing physicians that “supplementation with calcium and vitamin D is required in all patients except in cases of existing hypercalcemia”.

Hypocalcemia caused in this manner can lead to cardiac arrhythmias, and in particularly severe cases, can even be fatal if the hypocalcemia is not discovered and treated in time. Moreover, hypocalcemia often remains undetected because in outpatient clinical treatment, calcium levels are not measured at frequent intervals as a laboratory parameter. In order to prevent the therapy-induced adverse effect of hypocalcemia, the present invention proposes supplementation with calcium and vitamin D in the use of anti-RANKL antibodies that can prevent hypocalcemia.

An undetected deficiency of calcium and vitamin D can also negatively affect the entire outcome of antiresorptive therapy, e.g., with anti-RANKL antibodies, and thus reduce the desired protection against new fractures or even increase the number of fractures if calcium and vitamin D supplementation is not provided during antiresorptive therapy.

Osteonecrosis of the jaw is listed as an adverse effect of anti-RANKL. Osteonecrosis of the jaw may occur in treatment with anti-RANKL antibodies, and can lead to oral pain and non-healing wounds, leading to disintegration of the jaw.

As further adverse effects of antiresorptive therapy, cardiovascular adverse effects such as cardiac arrhythmias, cramps and secondary hyperparathyroidism may occur.

### SUMMARY

An aspect of the present invention is to solve the above problems and to provide novel pharmaceutical compositions of anti-RANKL antibodies and calcium and vitamin D and/or derivatives thereof in order to better treat disorders of bone metabolism or prevent these disorders, to provide an optimal effect, and to hinder or prevent therapy-induced adverse effects such as hypocalcemia and/or improper use.

In an embodiment, the present invention provides a method of administering a pharmaceutical composition comprising anti-RANKL antibodies, calcium and vitamin D to a patient in need thereof. The method includes subcutaneously administering the anti-RANKL antibodies at a dose of 60-180 mg in 1-3 ml of solution every four weeks, orally administering the calcium at a dose of 400 mg daily, and orally administering the vitamin D at a dose of 800-1200 IU daily. The patient in need thereof has skeletal-related complications due to solid tumors such as breast, prostate, lung, bowel, or bone cancer (osteosarcoma), pathological fractures, irradiation of the bone, spinal cord compression or bone surgery, bone metastases, pain in bone metastases, nerve entrapment, deformations due to one or more solid tumors such as breast cancer, prostate cancer, lung cancer, or multiple myeloma, and the administering of the pharmaceutical composition comprising anti-RANKL antibodies, calcium and vitamin D to the patient in need thereof at least one of treats and prevents hypocalcemia induced by anti-RANKL antibody therapy. The calcium and the vitamin D are provided as a combined single daily dose in a form of effervescent granules, swallowable tablets, swallowable capsules, chewable tablets, or ready-to-use granules.

### BRIEF DESCRIPTION OF THE DRAWINGS

The present invention is described in greater detail below on the basis of embodiments and of the drawings in which:

FIG. 1 shows a diagram of the calcium supply from the gastrointestinal tract into the blood and bone and influencing factors that increase or reduce calcium uptake;

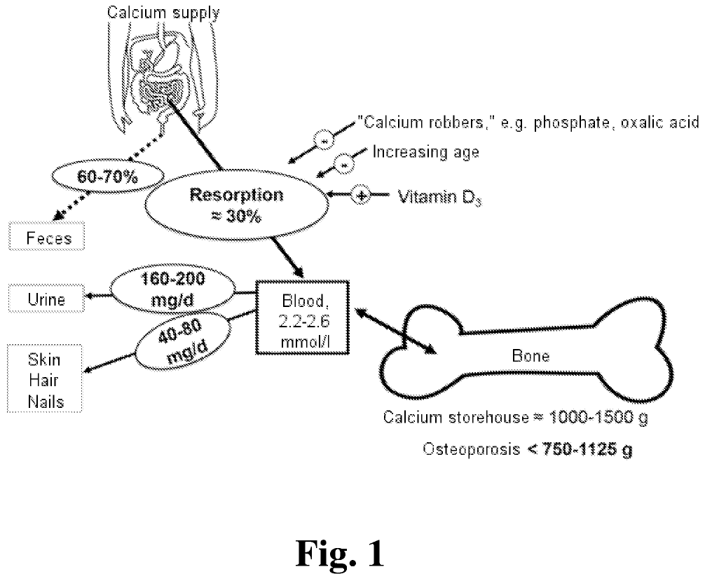

FIG. 2 shows the consequences of insufficient calcium uptake in the diet;

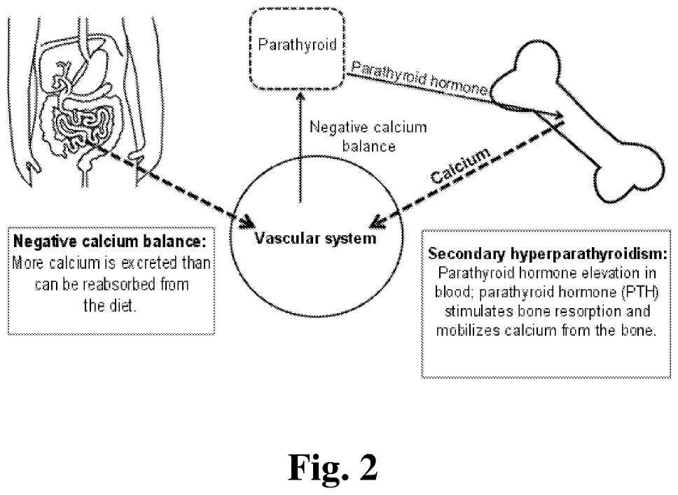

FIG. 3 shows the increase and decrease of bone mass over the course of a human life and the elevated fracture risk in old age with reduced bone mass;

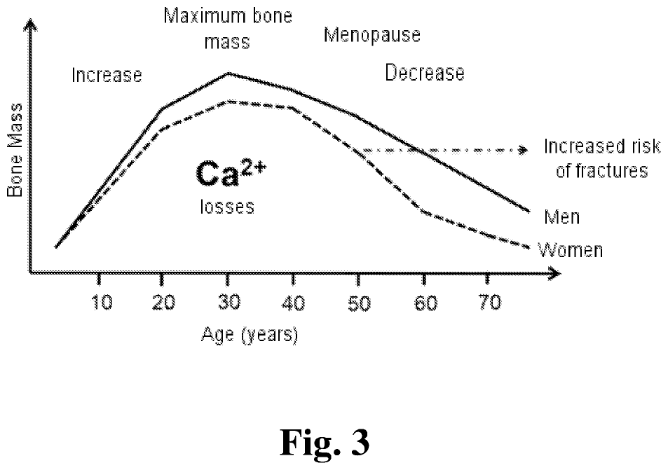

FIG. 4 shows the increased calcium requirement with increasing age due to reduced real calcium uptake;

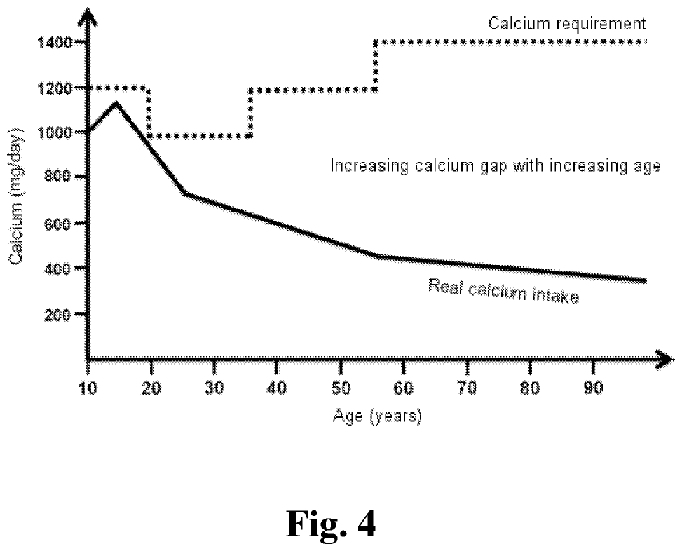

FIG. 5 shows that hypocalcemia is present if the total serum calcium is below 2.2 mmol/l (9 mg/dl) and that for the pharmaceutical inhibition of osteoclasts, the calcium concentration in the blood can only be balanced by the external supply of calcium (orally or intravenously;

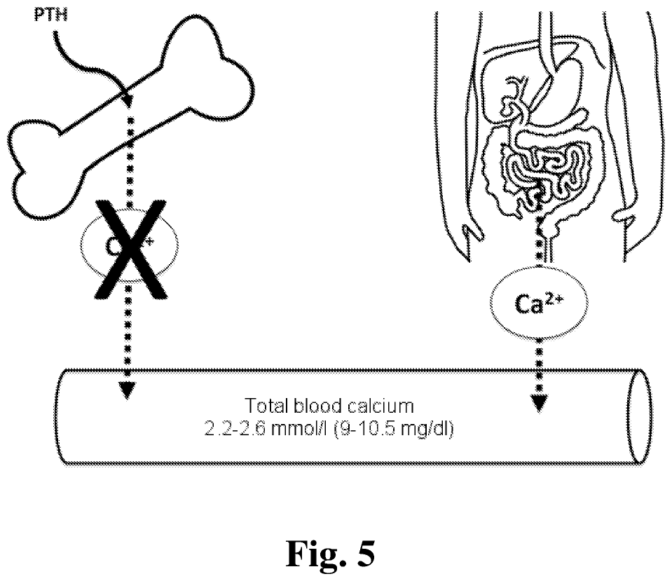

FIG. 6 shows an example of a protein sequence for RANKL (SEQ ID NO 7);

FIG. 7 shows an example of a cDNA sequence for the heavy chain of an anti-RANKL antibody (SEQ ID NO 1);

FIG. 8 shows an example of a protein sequence for the heavy chain of an anti-RANKL antibody (SEQ ID NO 2);

FIG. 9 shows an example of a DNA sequence for the light chain of an anti-RANKL antibody (SEQ ID NO 3);

FIG. 10 shows an example of a protein sequence for the light chain of an anti-RANKL antibody (SEQ ID NO 4);

FIG. 11 shows an example of a protein sequence for the variable region of the heavy chain of an anti-RANKL antibody (SEQ ID NO 5);

FIG. 12 shows an example of a protein sequence for the variable region of the light chain of an anti-RANKL antibody (SEQ ID NO 6);

FIG. 13 shows a further example of a protein sequence for the heavy chain of an anti-RANKL antibody (SEQ ID NO 8);

FIG. 14 shows a further example of a protein sequence for the heavy chain of an anti-RANKL antibody (SEQ ID NO 9); and

### SEQUENCE LISTING

The Sequence Listing associated with this application is filed in electronic form via EFS-Web and is hereby incorporated by reference into this specification in its entirety. The name of the text file containing the Sequence Listing is Karl_Sequence_Listing_Eng. The size of the text file is 20,044 Bytes, and the text file was created on Nov. 7, 2018.

### DETAILED DESCRIPTION

In a first embodiment, the present invention relates to the use of pharmaceutical compositions comprising anti-RANKL antibodies and/or antigen-binding fragments thereof and calcium and vitamin D for the treatment of osteoporosis, postmenopausal osteoporosis, manifest osteoporosis, corticoid-induced osteoporosis, osteoporosis in men or women, Paget's disease, and osteogenesis imperfecta for the prevention of fractures in the above-mentioned disorders, and for use in the treatment and/or prevention of hypocalcemia induced by anti-RANKL antibody therapy, characterized in that the pharmaceutical composition comprising anti-RANKL antibodies and/or antigen-binding fragments thereof is/are administered subcutaneously at yearly or six-month intervals, for example at a dose of 30 to 90 mg in 1-2 ml of solution (20-60 mg/ml), for example, 50-70 mg, for example, 60 mg, for example, in 1 ml of solution (60 mg/ml), and the pharmaceutical composition(s) comprising calcium is/are administered orally at a dose of 400-600 mg per day, for example, 500 mg per day, and vitamin D is administered orally at a dose of 800-1200 IU (international units) of vitamin D, for example, 1000 IU of vitamin D daily.

In a further embodiment, the present invention relates to the use of pharmaceutical compositions comprising anti-RANKL antibodies and/or antigen-binding fragments thereof and calcium and vitamin D for the prevention of skeletal-related complications, in particular pathological fractures, irradiation of the bone, spinal cord compression, bone surgery, bone metastases, pain in bone metastases, nerve entrapment or deformations due to one or more solid tumors such as e.g., breast cancer, prostate cancer, lung cancer, or multiple myeloma, and the prevention of fractures in the above-mentioned disorders, and for use in the treatment and/or prevention der hypocalcemia induced by anti-RANKL antibody therapy, characterized in that anti-RANKL antibodies and/or antigen-binding fragments thereof are administered subcutaneously, for example, at a dose of 60-180 mg in 1-3 ml of solution, for example, 80-150 mg, for example, 120 mg, in particular in 1.7 ml of solution (70 mg/ml) of anti-RANKL antibodies for at least 3-4 weeks, and 400-600 mg of calcium is orally administered daily, for example, 500 mg of calcium daily and 800-1200 IU of vitamin D daily, for example, 1000 IU of vitamin D daily.

The present invention moreover relates in further aspects to the use of various calcium compounds and vitamin D in the treatment and/or prevention of therapy-induced adverse effects, in the above-mentioned examples, and pharmaceutically suitable excipients and solvents.

Anti-RANKL antibodies are highly effective antiresorptive agents and are used for the treatment of disorders of bone metabolism. Despite their efficacy, treatment with anti-RANKL antibodies is accompanied by considerable, sometimes severe adverse effects. The present invention provides pharmaceutical compositions that substantially improve treatment with anti-RANKL antibodies with respect to efficacy and safety. In addition to anti-RANKL antibodies, the pharmaceutical compositions comprise calcium and vitamin D.

Anti-RANKL antibodies bind to RANKL. RANKL is a transmembrane protein, but can also occur as a soluble protein. RANKL plays a decisive role in the formation, function, and survival of osteoclasts. In multiple myeloma and bone metastases, increased osteoclast activity occurs that is stimulated by RANKL. This leads to increased bone resorption.

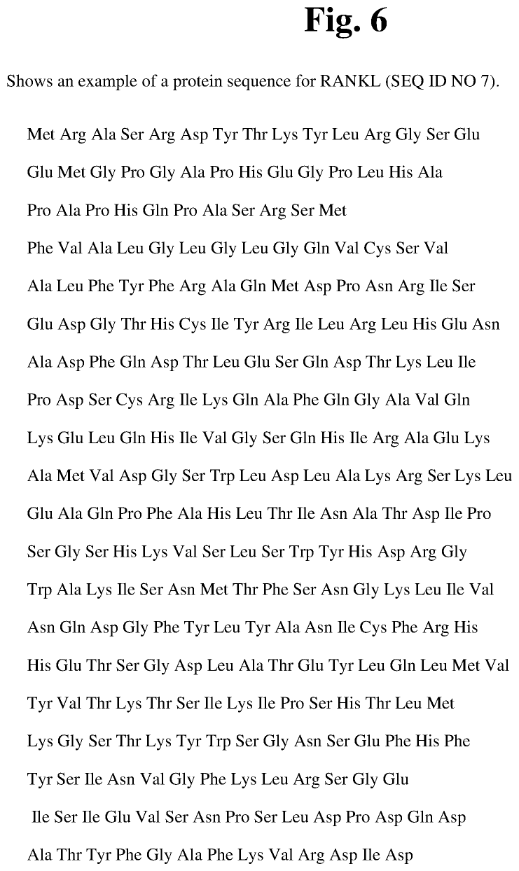

The sequence of the human RANKL protein is shown in FIG. 6. The protein is present in various isoforms. Isoform 2 lacks the amino acids 1-73, and isoform 3 lacks the amino acids 1-47.

Anti-RANKL antibodies prevent RANK-RANKL interaction. This reduces the number and the function of the osteoclasts and reduces bone resorption.

The term “anti-RANKL antibodies” includes all RANKL-binding molecules that bind at least one epitope of RANKL and in this way affect the activity of RANKL, i.e., block or reduce the binding of RANKL to RANK so that activation of the osteoclasts is reduced or inhibited. A selected human amino acid sequence of RANKL is shown in FIG. 6 (SEQ ID NO 7).

The term “epitope” refers to a binding site on an antigen, in this case RANKL, to which the anti-RANKL antibody specifically binds. The epitope binds to a primary amino acid sequence of RANKL that typically comprises at least 3 amino acids, ordinarily at least 5 or for example 8-10 amino acids. Depending on the 3-dimensional structure of RANKL, the epitope can also bind amino acids that do not follow one another in sequence, but are located in the vicinity because of the 3-dimensional structure.

The term “specifically binds” means that the antibody is capable of interacting with at least 2, for example, 3, and, for example, 4 amino acids of an epitope, for example, according to the “lock and key principle”.

The term “specific” means that the antibody binds to a RANKL molecule and essentially does not bind to other proteins or molecules. It may happen that the antibody cross-reacts with RANKL molecules of other species. The antibody should not, however, bind to other molecules, such as, for example, RANK. Binding is generally regarded as specific when the binding affinity is greater than 10⁻⁵ M. The specific binding affinity is, for example, approximately 10⁻¹¹ to 10⁻⁸ M (KD), for example, approximately 10⁻¹¹ to 10⁻⁹ M.

The term “essentially does not bind” means that the anti-RANKL antibody of the invention does not bind to any other protein, and in particular shows cross-reactivity of less than 30%, for example, 20%, for example, 10%, and in particular shows less than 9, 8, 7, 6, or 5% binding to another protein or molecule.

The term “polypeptide” has the same meaning as the term “protein”. Proteins contain one or more amino acids that are bonded to one another by covalent bonds. They may be subjected to post-translational modifications such as e.g., glycosylation. This is generally known in the art.

The term “antibody” includes a protein having one or a plurality of antigen-binding domains at least partially encoded by immunoglobulin genes or portions thereof. An “immunoglobulin” is an “antibody”. The antibody typically comprises glycosylated trimers (proteins) composed of 2 light chains of approximately 25 kDa each and 2 heavy chains of approximately 50 kDa each. Two types of light chains exist in antibodies: lambda and kappa. The light chains are composed respectively of one variable and one constant domain. These are referred to as VL and CL. Depending on the amino acid sequence of the constant region of the heavy chains, immunoglobulins are divided into 5 classes, A, D, E, G, and M, and some are divided into further subgroups, for example, IgG1, IgG2, IgG3, IgG4, IgA1, and IgA2. Each of the heavy chains has one domain, three variable domains for IgG and IgA, or four constant domains for IgM and IgE. Analogously, these are referred to as VH and CH1, CH2, CH3.

IgM antibodies comprise 10 antigen binding sites. The basic unit is composed of two heavy H chains and two light L chains. IgM is produced as the first immunoglobulin during an immune response and activates the complement system. Endogenous, naturally-produced IgM antibodies are secreted by B1 cells (CD20⁺, CD20⁺, CD27⁺, CD43⁺ and CD70⁺ cells). In addition to defending against penetrating microorganisms, they are involved in tissue homeostasis via clearance of apoptotic and modified cells by means of complement-dependent mechanisms. They inhibit inflammatory processes and remove modified cells.

IgA antibodies contain 2-5 of the 4 chain units that can form units together with the J chain. IgA can occur as a monomer (i.e., as only a single molecule) or as a dimer (two molecules joined at the long ends of the antibody y). In the case of the dimers, this is a so-called secretory IgA (sIgA).

Each light chain contains an N-terminal variable (V) domain (VL) and a constant domain (CL). Each heavy chain contains one N-terminal V domain (VH), 3 or 4 C domains (CHs), and one hinge region.

The constant domains of the antibody are not directly involved in the antigen binding, but may have various effector functions, for example, in antibody-dependent cellular toxicity. If an antibody exerts cellular toxicity, it should, for example, be the IgG1 subtype, while the IgG4 subtype does not have this capacity.

The binding of VH and VL forms an antigen-binding site. Each L chain is bound to the H chain via a covalent disulfide bridge, while the two H chains are bound to each other by one or several disulfide bridges, depending on the H chain isotype. The VH and VL domains are composed of four regions with relatively highly conserved sequences (FR1, FR2, FR3 and FR4) that form the framework for the three regions with hypervariable sequences, the complementary determining regions, CDRs. The CDRs are essentially responsible for the interaction between antigen and antibody. CDRs are more specifically designated CDR1, CDR2 and CDR3. Accordingly, CDR regions of the heavy chain are designated H1, H2, and H3, while the CDR regions of the light chain are designated L1, L2, and L3.

The term “variable” refers to the portion of the immunoglobulin domain that has variability in the sequence and defines the specificity and binding affinity of a certain antibody, such as e.g., variable domains. The variability is not distributed uniformly in the antibody, but is concentrated on the sub-domains of the respective light and heavy chains, which are designated hypervariable regions or CDRs.

The less conserved regions or the non-hypervariable regions of the variable domains are the so-called framework regions (FR), which often have a beta-folded structure. The FR regions and the CDRs form the structure that the antigen recognizes and binds (Kabat E. A., Wu, T. T., Perry, H., Gottesman, K. and Foeller, C. (1991) Sequences of Proteins of Immunological Interest, Fifth Edition. NIH Publication No. 91-3242). The constant domains are not directly involved in antigen-antibody binding, but mediate other reactions such as e.g., antibody-dependent and cell-mediated cytotoxicity and complement activation.

The term “CDR” or “CDRs” refers to the complementary determining region (CDR). As mentioned above, the CDRs are referred to as CDR 1-3, those of the light chain as CDRL1, CDRL2, and CDRL3 and those that contain the variable regions of the heavy chain as CDRH1, CDRH2 and CDRH3. CDRs make a decisive contribution to the activity of the antibody. The exact length of the CDRs is sometimes classified and numbered differently in the various systems. CDRs are designated in the following according to Kabat and Chothia. All systems have overlapping portions with respect to the designation of the hypervariable region within the variable sequence (Kabat et al., Chothia et al., J. Mol. Biol., 1987, 196; 901 and MacCallum et al., J. Mol. Biol., 1996, 262:732); in the present document, reference is primarily made to the numbering of Kabat.

The term “amino acid” or “amino acid residue” refers to amino acids as known in the art, selected from the group of alanine (Ala or A); arginine (Arg or R); asparagine (Asn or N); aspartate, (Asp or D); cysteine (Cys or C), glutamine (Gln or Q); glutamic acid (Glu or E); glycine (Gly or G); histidine (His or H); isoleucine (Ile or I); leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); valine (Val or V), wherein modified, synthetic, or rare amino acids can also be used.

The term “hypervariable region” or “CDR” refers to the segments of regions in the light (L chains) and heavy chains (H chains) of the immunoglobulins that show major differences in the amino acid sequence. They are the regions of an antibody or T cell receptor responsible for recognizing the antigen. The CDRs are the most variable parts of the receptors and are responsible for the diversity thereof. The three complementarity-determining regions CDR1, CDR2 and CDR3 are loops at the end of a V domain of antibodies. They come into direct contact with an antigen. At least two methods are known in the prior art for identifying CDRs: an approach based on cross-species sequence variability (Kabat et al.), and another approach based on crystallographic studies of the antigen-antibody complex (Chothia, C. et al., J. Mol. Biol., 196:901-917 (1987)). It is generally preferred to carry out CDR identification in accordance with the so-called Kabat numbering.

The term “framework region” is known to the person having ordinary skill in the art and refers to the parts of the variable region of the antibody present between the hypervariable regions, the CDRs. Such framework regions are typically designated framework regions 1-4 (FR1, FR2, FR3 and FR4) and form the framework for the 6 CDRs (three of the light chain and three of the heavy chain) in the three-dimensional space in order to provide an antigen-binding surface.

The present invention includes all anti-RANKL that are considered to be interchangeable according to the Kabat numbering system and have an antigen-binding function, i.e., can bind to a RANKL epitope.

“Antibody” means a monoclonal, polyclonal, monospecific, bispecific, bifunctional, single-chain, synthetic, recombinant, mutated, human, humanized, or chimeric antibody (Harlow and Lane, “Antibodies, A Laboratory Manual”, CSH Press, Cold Spring Harbor, USA) that binds to a RANKL molecule or a derivative that retains or essentially retains this binding capacity. Domain antibodies (dAbs) and nanobodies are also included in the term “antibody”.

A bispecific or bifunctional antibody is an artificial hybrid antibody with two different heavy/light chains and two different binding sites. The term and the methods for production thereof are known to the person having ordinary skill in the art (e.g., Songsivilai & Lachmann, Clin. Exp. Immunol. 79:315-321 (1990); Kostelny et al., J. Immunol. 148, 1547-1553 (1992).

The production of monoclonal antibodies is known in the prior art (e.g., Kohler and Milstein (1975), Nature, 256:495-499); further methods include so-called phage display (Ladner et al., U.S. Pat. No. 5,223,409).

Derivatives of these antibodies can, for example, be chimeric antibodies that, for example, comprise a variable region of the mouse or rat and a human constant region. The term “antibody” also includes a bifunctional or bispecific antibody and antibody constructs such as Fvs (scFv) composed of individual chains or antibody fusion proteins. The term “scFv” (single chain Fv fragment) is known to the person having ordinary skill in the art and is preferred because the fragment can be recombinantly produced.

The antibody can be human or humanized. The term “humanized antibody” means that at least one antibody binding site (complementary determining region (CDR)) such as e.g., CDR3, and, for example, all 6 CDRs, are replaced by CDRs of a human antibody of the desired specificity. The non-human constant region(s) of the antibody are optionally replaced by the constant region(s) of a human antibody. Methods for the production of humanized antibodies are described, for example, in EP 0239400 A1 and WO 90/07861.

A human antibody is alternatively provided to the humanized antibody. Transgenic animals such as e.g., mice are capable after immunization of producing human antibodies without typical mouse antibody sequences. They can form complete human antibodies (e.g., Jakobovits et al., Proc. Natl. Acad. Sci. USA, 90:2551 (1993)). So-called phage display technology can alternatively be used for producing human antibodies or antigen-binding fragments. The techniques are known in the prior art (McCafferty et al., Nature 348: 552-553 (1990); Johnson, Kevin S. and Chiswell, David J., Curr. Opin. Struct. Biol. 3: 564-571 (1993)).

The term antigen-binding fragment refers to a fragment of an “antibody” as the term is defined above, such as e.g., separate light and heavy chains, Fab, Fab/c, Fv, scFv, Fd, dAb, Fab′, F(ab′)2. An antigen-binding fragment can comprise a variable region of the light chain and a variable region of the heavy chain, not necessarily both at the same time.

Methods for producing and isolating antibodies are known in the prior art, see e.g., Harlow and Lane; Antibodies: A Laboratory Manual, Cold Spring Harbor Laboratory, New York (1988). General antibody purification methods include salt precipitation (for example, with ammonium sulfate); ion-exchange chromatography (for example, on a cation or anion exchange column that is allowed to run at neutral pH and eluted with step gradients of increasing ion strength); gel filtration chromatography (including gel filtration HPLC) and chromatography on affinity resins such as e.g., protein A, protein G, hydroxyapatite or anti-Ig. Antibodies can also be purified on affinity columns containing an antigen segment. Preferred fragments are purified by protein A CL Sepharose 4B chromatography followed by chromatography on a DEAE Sepharose 4B ion exchange column.

The present invention also includes hybrid antibodies in which one pair of H and L chains is obtained from a first antibody, while the other pair is obtained from H and L chains from another second antibody. For the purposes of the present invention, a pair of L and H chains is derived from anti-RANKL. In an example, each L-H chain pair binds various epitopes of a RANKL-specific antigen. Such hybrids can also be formed using humanized H or L chains. The present invention also includes other bispecific antibodies, such as e.g., those containing two separate antibodies that are covalently bonded via their constant regions.

The size of the antigen-binding fragment can be only the minimum size required to provide a desired function. It can optionally comprise a further amino acid sequence, either native to the antigen-binding fragment or from a heterologous source as desired. Anti-RANKL antigen-binding fragments can comprise 5 successive amino acid residues from an antibody V region sequence. Polypeptides are also included that comprise 7 amino acid residues, for example, 10 amino acid residues, for example, 15 amino acid residues, for example, 25 amino acid residues, for example, 50 amino acid residues, for example, 75 amino acid residues from the antibody L or H chain V region. Polypeptides comprising the entire antibody L or H chain V region can, for example, be used.

Substitutions can range from changing or modifying one or more amino acid residues to the completely new design of a region such as e.g., the V region. Amino acid substitutions, if present, can, for example, be conservative substitutions that do not negatively affect the folding or functional properties of the peptide. Groups of functionally related amino acid residues within which conservative substitutions can be made are glycine/alanine, valine/isoleucine/leucine; asparagine/glutamine, aspartic acid/glutamic acid; serine/threonine/methionine; lysine/arginine and phenylalanine/tyrosine/tryptophan. Antibodies may be glycosylated or unglycosylated, be modified after translation (e.g., acetylation and phosphorylation), or be synthetically modified (e.g., binding of a labeling group).

Anti-RANKL antibodies are also provided that have changes in their amino acid sequence. These changes can include deletions, insertions, and substitutions of individual or multiple amino acids. Because of this, post-translational modifications may also be subjected to changes.

Changes in the CDRs of the light and heavy chain are most useful, particularly in the hypervariable regions, but FR changes in the light and/or the heavy chain can also be considered. If a CDR sequence comprises 6 amino acids, 1-3 amino acids can be changed. If it comprises 15 amino acids, 1-6 amino acids can be changed. If CDRs are changed in the light and/or the heavy chain, the changed amino acid sequence should be at least 60%, for example 65%, for example, 70%, for example, 75%, and, for example, 80% identical with the original CDR sequence. For this reason, the degree of identity with the original CDR depends on the length of the respective CDRs. While a CDR with 5 amino acids and one change is 80% identical, the change in a longer CDR with one change in an amino acid is lower on a percentage basis. CDRs can therefore have differing degrees of identity with the original CDR, e.g., CDRH1 can be 90% identical while CDRL2 is 83% identical.

Examples of changes in the anti-RANKL antibody are shown in Table 1.

TABLE 1

| Original | | Preferred |
|---|---|---|
| Amino Acid | Example Substitutions | Substitutions |
| Ala | Val; Leu; Ile | Val |
| Arg | Lys; Gln; Asn | Lys |
| Asn | Gln; His; Asp; Lys; Arg | Gln |
| Asp | Glu; Asn | Glu |
| Cys | Ser; Ala | Ser |
| Gln | Asn; Glu | Asn |
| Glu | Asp; Gln | Asp |
| Gly | Ala | Ala |
| His | Asn; Gln; Lys; Arg | Arg |
| He | Leu; Val; Met; Ala; Phe | Leu |
| Leu | Norleucin; Ile; Val; Met; Ala | Ile |
| Lys | Arg; Gln; Asn | Arg |
| Met | Leu; Phe; Ile | Leu |
| Phe | Leu; Val; Ile; Ala; Tyr | Tyr |
| Pro | Ala | Ala |
| Ser | Thr | Thr |
| Thr | Ser | Ser |
| Trp | Tyr; Phe | Tyr |
| Tyr | Trp; Phe; Thr; Ser | Phe |
| Val | Ile; Leu; Met; Phe; Ala | Leu |

These are listed as examples and can, for example, be 60%, for example, 65%, for example, 70%, for example, 75%, and, for example, 80% identical to the original CDR sequence. The sequences are included that bind specifically to RANKL. The methods concerning changes in the CDRs are moreover known (e.g., U.S. Pat. Nos. 6,180,370; 5,693,762; 5,693,761; 5,585,089; and 5,530,101).

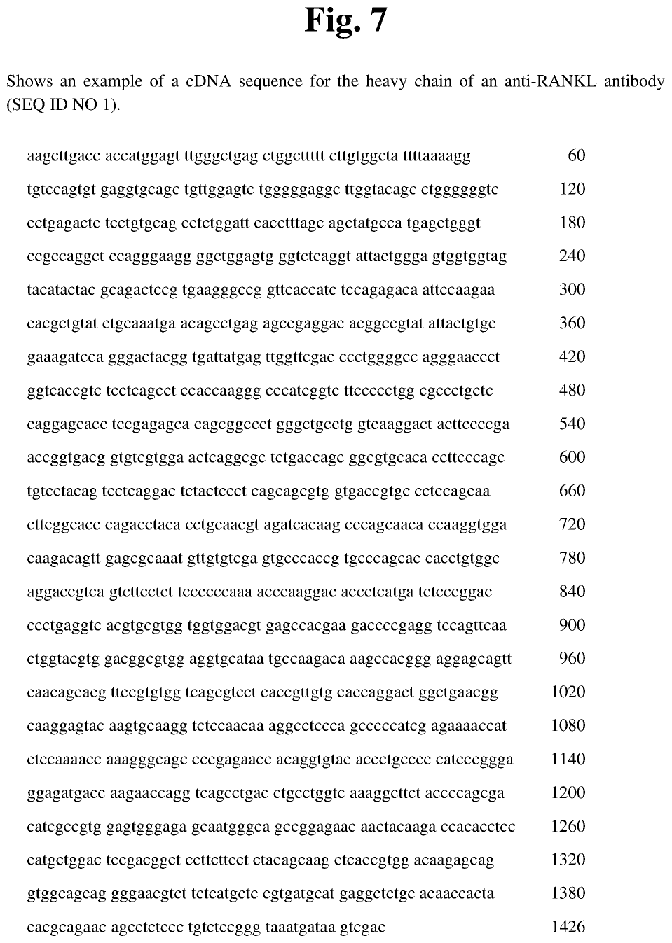

An example of a cDNA sequence for the heavy chain of an anti-RANKL antibody is shown in FIG. 7 (SEQ ID NO 1).

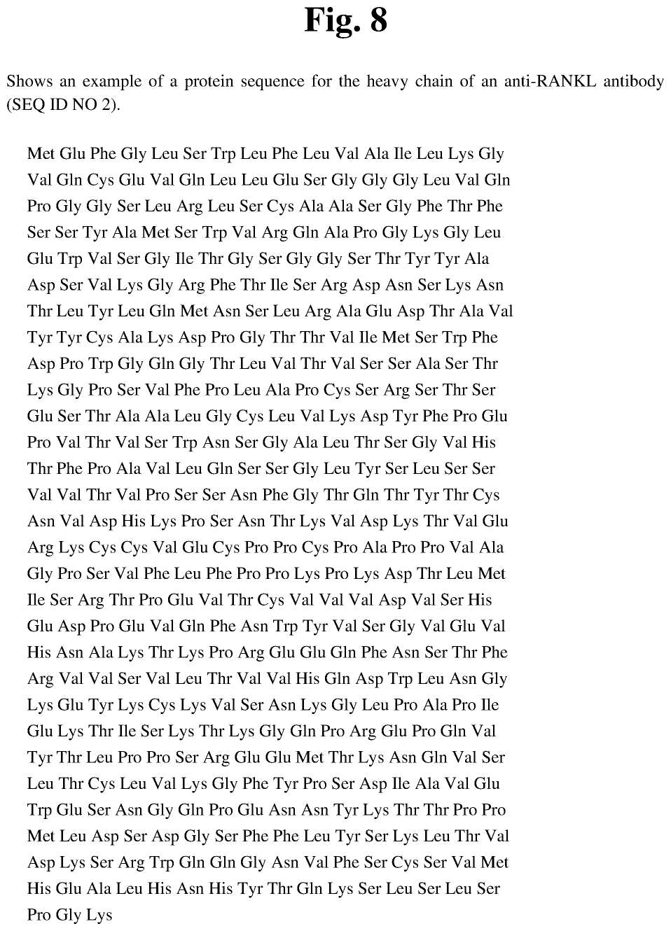

An example of a protein sequence for the heavy chain of an anti-RANKL antibody is shown in FIG. 8 (SEQ ID NO 2).

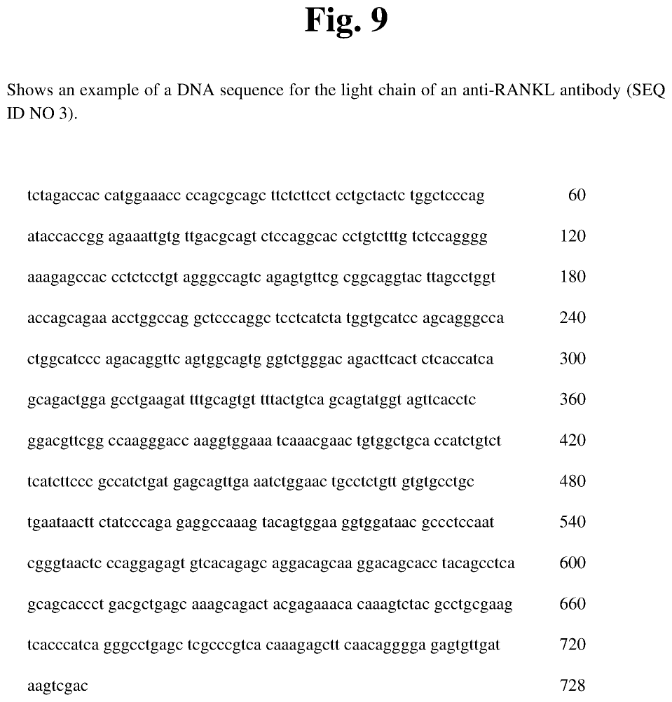

An example of a DNA sequence for the light chain of an anti-RANKL antibody is shown in FIG. 9 (SEQ ID NO 3).

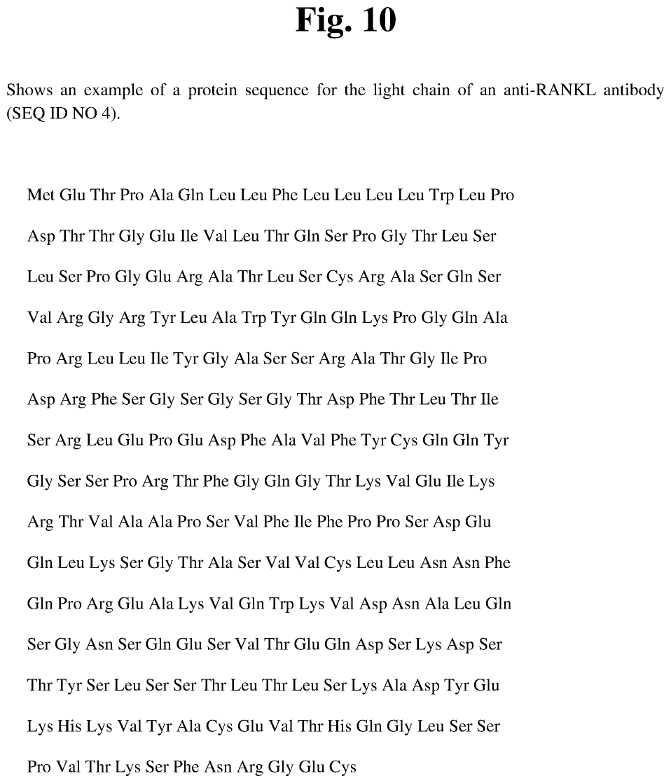

An example of a protein sequence for the light chain of an anti-RANKL antibody is shown in FIG. 10 (SEQ ID NO 4).

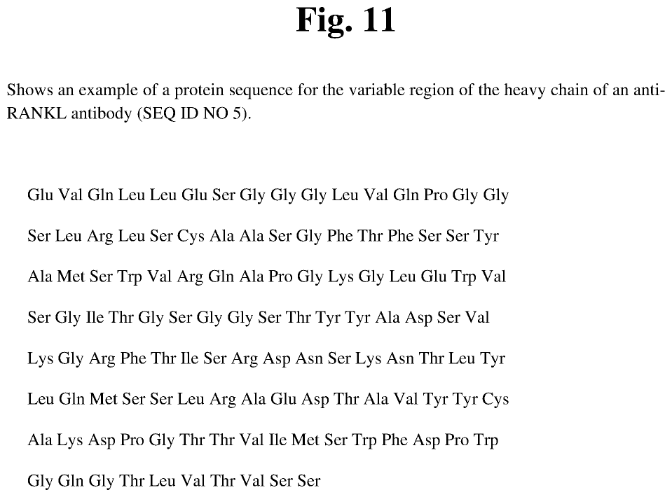

An example of a protein sequence for the variable region of the heavy chain of an anti-RANKL antibody is shown in FIG. 11 (SEQ ID NO 5).

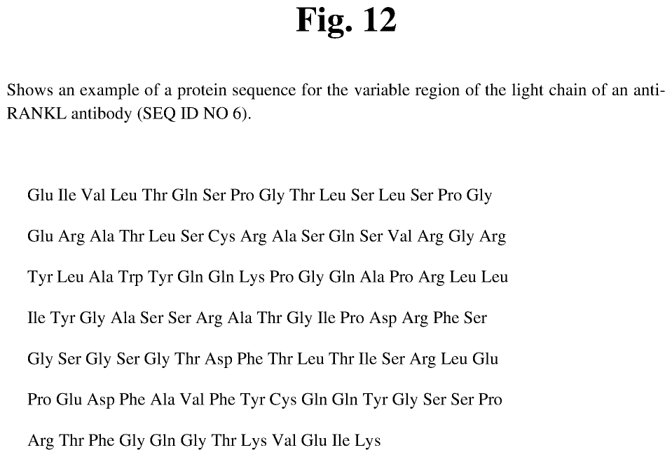

An example of a protein sequence for the variable region of the light chain of an anti-RANKL antibody is shown in FIG. 12 (SEQ ID NO 6).

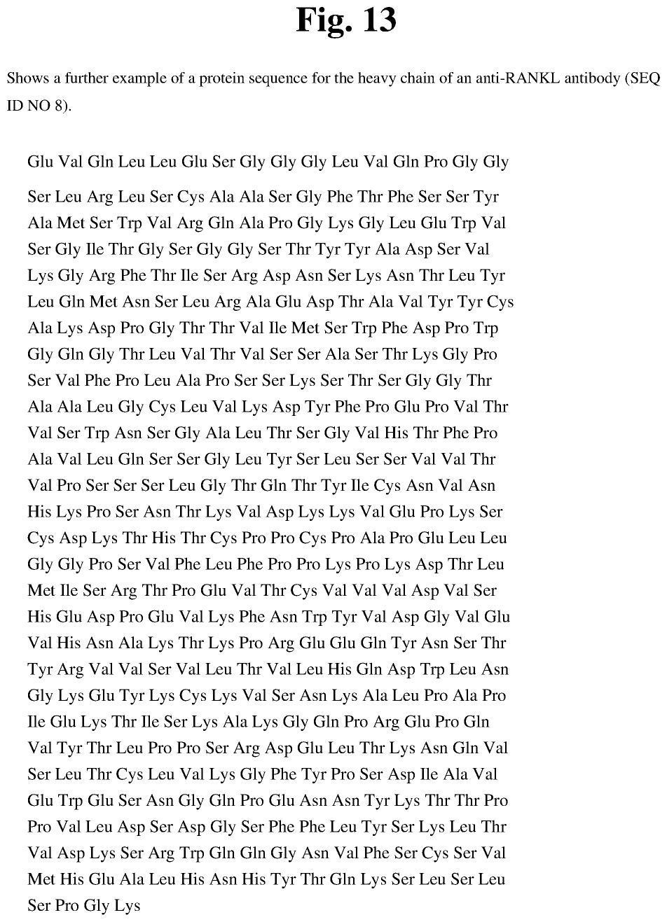

An example of a protein sequence for RANKL is shown in FIG. 13 (SEQ ID NO 8).

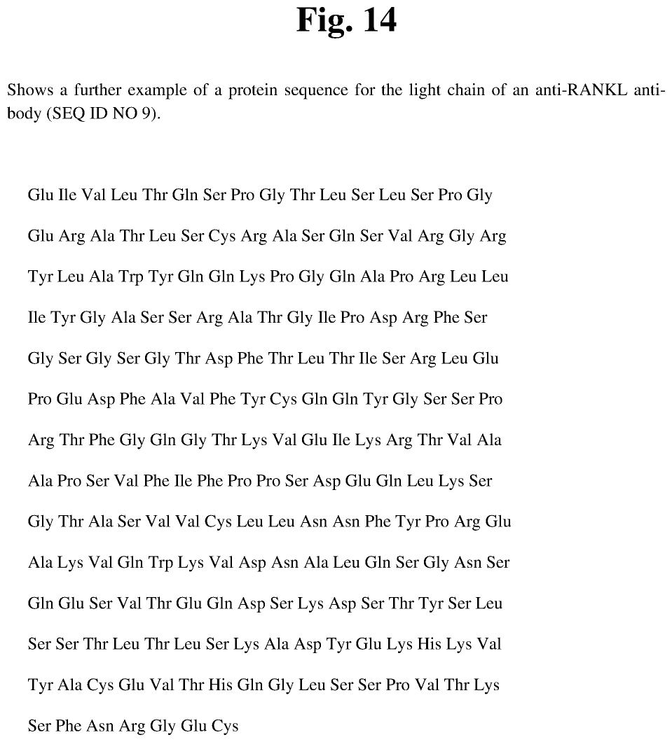

A further example of a protein sequence for the heavy chain of an anti-RANKL antibody is shown in FIG. 14 (SEQ ID NO 9).

According to the present invention, the antigen-binding fragments of the anti-RANKL antibody are also included.

Prolia (contains denosumab, an anti-RANKL antibody that is approved as a medicinal preparation) is marketed by Amgen GmbH. It contains 60 mg of denosumab injection solution. Each prefilled syringe contains 60 mg of denosumab in 1 ml of solution (60 mg/ml). The recommended dose of Prolia is 60 mg, which is administered as a single subcutaneous injection once every 6 months (thigh, abdomen, or upper arm). Prolia is used for the treatment of osteoporosis in postmenopausal women for the prevention of vertebral and non-vertebral fractures, for treatment to increase bone mineral density in men with osteoporosis and increased fracture risk, and as concomitant treatment in women with breast cancer under adjuvant treatment with aromatase inhibitors and in men with prostate cancer under hormone ablation therapy if there is increased fracture risk.

Xgeva, medicinal preparation, also contains anti-RANKL antibody (denosumab, Amgen, each injection vial contains 120 mg of denosumab in 1.7 ml of solution (70 mg/ml)). Xgeva is used for the prevention of skeletal-related complications (pathological fracture, irradiation of the bone, spinal cord compression or bone surgery) in adults with bone metastases due to solid tumors and in the treatment of adults and skeletally mature young people with giant cell tumors of the bone that are not resectable or in which surgical resection is likely to lead to serious morbidity. The summary of product characteristics only recommends sufficient intake of calcium and vitamin D, and the exact dosage is not indicated.

The sufficient daily total calcium supply is 1000 mg. Calcium is absorbed via the diet, so that the required daily dose can be achieved by consuming a calcium-rich diet. A calcium intake of more than 2500 mg per day is considered to be problematic, among other reasons because this can increase the risk of kidney stone formation. Because of various reports of cardiovascular adverse effects due to excessive calcium intake, many patients have refrained from taking in calcium in sufficient amounts, even during treatment with anti-RANKL antibodies.

This in many cases endangers the result of treatment with anti-RANKL antibodies as a whole and causes cardiovascular adverse effects due to the insufficient calcium supply or due to strong inhibition of bone resorption by treatment with anti-RANKL antibodies, which inhibits resorption of calcium from the bone into the blood.

This leads to hypocalcemia, which results in cardiovascular adverse effects. This in principle causes exactly the opposite of the desired result. Because of the calcium deficiency, not the excess supply of calcium, increased cardiovascular adverse effects now occur that could have been prevented by providing an adequate calcium supply during treatment with anti-RANKL antibodies.

Supplementation with a pharmaceutical composition containing 1000-1500 mg of calcium per day in persons consuming a diet containing sufficient calcium will lead to an excess intake of calcium, which increases the risk of cardiovascular adverse effects. In contrast to the study designs, which in the summary of product characteristics of Aclasta contain a daily dose of 1000-1500 mg of calcium, a lower dosage is provided in the present invention in order to treat or prevent hypocalcemia and cardiovascular adverse effects.

The amount of calcium in the pharmaceutical composition can, for example, be 400-600 mg of calcium per day, for example, 500 mg of calcium per day, in order to achieve a basic level of calcium in patients with a calcium-poor diet, and to avoid an excessive supply of calcium, which is associated with the above-mentioned risks and is therefore unacceptable to patients. According to the results report of the National Consumption Study II carried out in Germany, 46% of men and 55% of women do not take in the recommended daily amount of calcium. In the age group from 51-80 years of age, the average calcium intake is in the range of 490-1790 mg of calcium per day.

According to the summary of product characteristics of Xgeva, 500 mg of calcium should be taken in per day, but in combination with 400 IU of vitamin D per day. The studies for Xgeva were geared toward US patients. In the US, many foods are supplemented with vitamin D, such as milk, orange juice, various breads, or breakfast cereals. This is not generally the case in Germany and in the EU so that, according to the present invention, a larger amount of vitamin D of 800-1200 IU per day is provided, for example, 1000 IU of vitamin D per day. The higher amount of vitamin D is capable of promoting the transport of a potentially smaller amount of calcium from the gastrointestinal tract into the blood and thus achieving a sufficient serum calcium level despite the smaller amount of calcium.

In contrast to the summary of product characteristics for Xgeva, therefore, a combined therapy with anti-RANKL antibodies, 400-600 mg of calcium, and 800-1200 IU of vitamin D daily is provided for the prevention and treatment of hypocalcemia. The increased vitamin D provides that the low calcium dose of 400-600 mg, for example, 500 mg of calcium daily, maintains the calcium concentration at the standard level above 2.2 mmol/1 (9 mg/dl).

The vitamin D level can be easily measured. The combination according to the present invention is provided for a vitamin D serum concentration that is not below 20 ng/ml (50 nmol/1 of serum).

In a severe deficiency (<10 ng/ml of serum), substitution should be given with 200,000 IU over 10 days and then 20,000 IU weekly. In a marked deficiency (10-20 ng/ml of serum), initial substitution should be given with 100,000 IU, followed by maintenance of 20,000 IU/week. In the case of an ordinary deficiency (21-30 ng/ml of serum), substitution of 20,000 IU/week should be carried out. When the normal vitamin D level is reached (31-60 ng/ml of serum), daily substitution according to the present invention of 800-1200 IU, for example, 1000 IU of vitamin D, should be given.

Supplementation with 400 IU of vitamin D is not sufficient to maintain the vitamin D level in the normal range of 31-60 ng/ml of serum in administration of the pharmaceutical composition according to the present invention of anti-RANKL antibodies and calcium.

In this manner, via the pharmaceutical composition according to the present invention of anti-RANKL antibodies, calcium, and vitamin D, hypocalcemia is prevented.

According to the present invention, the pharmaceutical composition comprising anti-RANKL antibodies for the treatment of osteoporosis, postmenopausal osteoporosis, manifest osteoporosis, corticoid-induced osteoporosis, osteoporosis in men or women, Paget's disease, osteogenesis imperfecta, for the prevention of fractures in the above-mentioned disorders, and for use in the treatment and/or prevention of hypocalcemia induced by anti-RANKL antibody therapy, is administered subcutaneously at yearly or six-month intervals, for example, at a dose of 30 to 90 mg in 1-2 ml of solution (20-60 mg/ml), for example, 50-70 mg, for example, 60 mg, for example, in 1 ml of solution (60 mg/ml).

According to the present invention, the pharmaceutical composition comprising anti-RANKL antibodies is subcutaneously administered in the field of oncology for the treatment and/or prevention of skeletal-related complications, in particular due to solid tumors, particularly breast, prostate, lung, bowel, or bone cancer (osteosarcoma), pathological fractures, irradiation of the bone, spinal cord compression or bone surgery, bone metastases, pain in bone metastases, nerve entrapment, deformations due to one or more solid tumors such as e.g., breast cancer, prostate cancer, lung cancer, or multiple myeloma, and the prevention of fractures in the above-mentioned disorders and for use in the treatment and/or prevention of hypocalcemia induced by anti-RANKL antibody therapy, for example, at a dose of 60-180 mg in 1-3 ml of solution, for example, 80-150 mg, for example, 120 mg, in particular in 1.7 ml of solution (70 mg/ml) of anti-RANKL antibodies, for at least 3-4 weeks.

According to the present invention, the above-listed pharmaceutical compositions are used, wherein the anti-RANKL antibody comprises a monoclonal, polyclonal, monospecific, bispecific, bifunctional, single-chain, synthetic, recombinant, mutated, human, humanized, chimeric, IgG, IgA, IgM or IgE antibody, an antigen-binding fragment or an antibody construct such as Fvs (scFv) composed of individual chains or antibody fusion proteins or a fragment thereof, in particular separate light and heavy chains, Fab, Fab/c, Fv, scFv, Fd, dAb, Fab′ or F(ab′)2, wherein a fragment comprises a variable region of the light chain and/or a variable region of the heavy chain.

The term “calcium” includes all known pharmaceutically acceptable calcium compounds, including the following in particular, without being limited thereto: calcium citrate, further designations: tricalcium citrate, TCC, tricalcium dicitrate, tricalcium-di-[2-hydroxy-1,2,3-propanetricarboxylate]-tetrahydrate, E 333, C₁₂H₁₀Ca₃O₁₄, CAS numbers: 813-94-5 (anhydrous), 5785-44-4 (tetrahydrate); calcium gluconate monohydrate (C₁₂H₂₂CaO₁₄. H₂O, CAS numbers: 299-28-5 (anhydrous) and 66905-23-5 (monohydrate)); calcium lactate gluconate, a double salt of lactic and gluconic acid in the form of a mixture, further designations: CLG, calcium lactate gluconate, calcium lactogluconate, E 327, E 578, CAS numbers: 11116-97-5, 814-80-2 (calcium lactate pentahydrate), 18016-24-5 (calcium gluconate monohydrate), or calcium carbonate CaCO₃, CAS number: 471-34-1, calcium phosphate, calcium dihydrogen phosphate, calcium hydrogen phosphate, calcium hydrogen phosphate dihydrate, calcium acetate, calcium ascorbate, calcium chloride, calcium glucoheptonate, calcium glycerophosphate and/or calcium sulfate.

The term “vitamin D” includes vitamin D or derivatives thereof, in particular, without being limited thereto, vitamin D3 (cholecalciferol, C₂₇H₄₄O, CAS number: 67-97-0), calcitriol (1,25-dihydroxyvitamin D3, 1α,25-dihydroxycholecalciferol, 1,25(OH)₂ vitamin D3, 1,25(OH)₂D3, (5Z,7E)-(1S,3R)-9,10-secocholesta-5,7,10(19)-triene-1,3,25-triol, CAS number: 32222-06-3) or 1α,25-dihydroxycholecalciferol (biologically active form of vitamin D3), alfacalcidol (1α-hydroxyvitamin D3), 24,25-dihydroxyvitamin D3 or calcifediol (25-hydroxyvitamin D3 25-hydroxycholecalciferol, CAS number: 19356-17-3, IUPAC name: (6R)-6-[(1R,3aR,4E,7aR)-4-[(2Z)-2-[(5S)-5-hydroxy-2-methylidene-cyclohexylidene]ethylidene]-7a-methyl-2,3,3a,5,6,7-hexahydro-1H-inden-1-yl]-2-methyl-heptan-2-ol), vitamin D2 (3β,5Z,7E,22E)-9,10-secoergosta-5,7,10(19),22-tetraen-3-ol, calciferol, ergo-calciferol, CAS number: 50-14-6 or biological forms thereof.

Both vitamin D preparations and anti-RANKL antibodies (denosumab, Prolia and Xgeva, Amgen Inc.) and calcium are commercially available, and the methods for the production thereof are known to the person having ordinary skill in the art.

In a further example, the pharmaceutical compositions are suitable for use in the treatment and/or prevention of adverse effects of the anti-RANKL antibodies, in particular osteonecrosis of the jaw accompanied by oral pain, non-healing wounds leading to disintegration of the jaw, hypocalcemia, cardiovascular adverse effects such as cardiac arrhythmias, cramps, and secondary hyperparathyroidism.

In an example, the above-indicated amounts of anti-RANKL antibodies are provided in an infusion solution of 1-3 ml in a pharmaceutically acceptable solution such as, for example, isotonic saline solution or isotonic sorbitol-sodium acetate-polysorbate solution, isotonic sorbitol-sodium acetate solution, isotonic glucose solution, or other pharmaceutically suitable isotonic solutions of suitable pH.

In a further example, calcium and vitamin D are provided with pharmaceutically suitable excipients, in particular lactose, starch, acidifying agents, in particular citric acid and malic acid, acid regulators, in particular sodium hydrogen carbonate and sodium carbonate, humectants, in particular sorbitol, xylitol and inulin, separating agents, in particular tricalcium phosphate, fatty acids, in particular magnesium salts, in particular magnesium stearate, natural and nature-identical and other flavoring and flavoring substances, sweeteners, in particular sodium cyclamate, aspartame and sodium saccharin, maltodextrin, dyes, in particular red beet juice powder and tiboflavin-5′-phosphate, silicon dioxide, in particular highly disperse, silicon dioxide hydrate, phenylalanine, gum arabic, saccharose, gelatins, cornstarch, soy oil, glycerol, DL-alpha tocopherol, isomalt, sodium hydrogen carbonate, sodium dihydrogen carbonate, sodium citrate, sodium dihydrogen citrate, carmellose sodium, acesulfame potassium, sodium ascorbate, triglycerides, in particular medium-chain, and/or in pharmaceutically compatible liquids, in particular water, isotonic saline or glucose solution.

In a further example, anti-RANKL antibodies, calcium and vitamin D, optionally with pharmaceutically suitable excipients and/or liquids, are provided in particular as effervescent tablets, swallowable tablets or capsules, chewable tablets, effervescent granules, ready-to-use granules, drinking solutions, drops, sublingual sprays, as infusion solution concentrates, instant infusions, injection solution concentrates, injection solutions, or prefilled syringes, either individually or in combination.

The following figures and examples are for explanatory purposes, but do not limit the invention to said figures or examples.

### Example 1

Vitamin D deficiency is a common diagnosis in osteology practice. At the beginning of therapy, 89 of 423 patients showed a vitamin D deficiency that was below the standard value of 20 ng/ml of serum or 50 nmol/l. The maintenance dose was 1000 IU of vitamin D daily. A maintenance dose of 400 IU of vitamin D daily was not sufficient to maintain the vitamin D concentration at the standard level. Table 2 shows that a maintenance dose of 1000 IU of vitamin D is suitable for maintaining the vitamin D level in the normal range:

TABLE 2

| | | |
|---|---|---|
| | | 25(OH) vitamin D3 |
| | 25(OH) vitamin D3 | serum concentration |
| | serum | at maintenance dose |
| | concentration | of 1000 IU of |
| Patient | initial value | vitamin D daily |

| | | | | |
|---|---|---|---|---|
| 85 year-old female | 9.4 | ng/ml | 25.5 | ng/ml |
| 65 year-old female | 8.8 | ng/ml | 23.4 | ng/ml |
| 77 year-old female | 12.4 | ng/ml | 29.2 | ng/ml |
| 81 year-old female | 10.4 | ng/ml | 30.4 | ng/ml |
| 75 year-old female | 14.6 | ng/ml | 28.8 | ng/ml |
| 86 year-old female | 10.9 | ng/ml | 23.7 | ng/ml |
| 81 year-old female | 8.6 | ng/ml | 23.2 | ng/ml |
| 71 year-old female | 17.1 | ng/ml | 30.5 | ng/ml |

### Example 2

The vitamin D level of 37 further patients was analyzed. At the beginning of therapy, a vitamin D deficiency [25(OH)Vit D serum concentration <30 ng/ml of serum or 75 nmol/l] was present in approx. 51% of the patients (n=19). In 27% of the patients (n=10), the 25(OH)Vit D serum concentration was below 20 ng/ml of serum or 50 nmol/1 (=severe vitamin D deficiency). The respective left columns of Table 3 below show the daily intake of calcium in the diet in mg. The daily intake of less than 1000 mg in the diet results in a negative calcium balance. This gives rise to secondary hyperparathyroidism, and bone tissue is lost. A daily supply of less than 500 mg of calcium is associated with an increased fracture risk for non-vertebral fractures.

TABLE 3

| Oncology | | | Osteoporosis | | | All Patients | | |
|---|---|---|---|---|---|---|---|---|
| (n = 38) | | | (n = 11) | | | (n = 49) | | |
| 74 | 21% | 74% | 177 | 18% | 64% | 74 | 20% | 73% |
| 230 | | | 496 | | | 177 | | |
| 257 | | | 738 | 46% | | 230 | | |
| 314 | | | 800 | | | 257 | | |
| 345 | | | 819 | | | 314 | | |
| 359 | | | 872 | | | 345 | | |
| 435 | | | 904 | | | 359 | | |
| 449 | | | 1136 | 27% | 36% | 435 | | |
| 558 | 53% | | 1300 | | | 449 | | |
| 667 | | | 1372 | | | 496 | | |
| 702 | | | 1866 | 9% | | 558 | 53% | |

| | |
|---|---|
| 711 | 667 |

| | | | | | | |
|---|---|---|---|---|---|---|
| 746 | | | 1) More than 70% <1,000 | 702 | | |
| 768 | | | mg of calcium/day | 711 | | |
| 776 | | | <1,000 mg of calcium/day | 738 | | |
| 788 | | | constitutes a negative | 746 | | |
| 792 | | | calcium balance and sec. | 768 | | |
| 795 | | | hyperparathyroidism | 776 | | |
| 831 | | | 2) Approx. 20% <500 | 788 | | |
| 866 | | | mg of calcium/day | 792 | | |
| 881 | | | <500 mg of calcium/day | 795 | | |
| 883 | | | constitutes an increased | 800 | | |
| 909 | | | risk of vertebral | 819 | | |
| 910 | | | fractures (DVO | 831 | | |
| 952 | | | LL 2014) | 866 | | |
| 958 | | | | 872 | | |
| 958 | | | | 881 | | |
| 979 | | | | 883 | | |
| 1050 | 18% | 26% | | 904 | | |
| 1135 | | | | 909 | | |
| 1203 | | | | 910 | | |
| 1215 | | | | 952 | | |
| 1250 | | | | 958 | | |
| 1264 | | | | 958 | | |
| 1449 | | | | 979 | | |
| 1538 | 8% | | | 1050 | 20% | 27% |
| 1706 | | | | 1135 | | |
| 1763 | | | | 1136 | | |

| | |
|---|---|
| 1203 | |
| 1215 | |
| 1250 | |
| 1264 | |
| 1300 | |
| 1372 | |
| 1449 | |
| 1538 | 7% |
| 1706 | |
| 1763 | |
| 1866 | |

### Example 3

The so-called “chair-rising test” (standing up test) was carried out in 3 patients.

The chair-rising test (standing up test) allows a determination to made concerning the strength and falling risk of the test person. The test person is asked to stand up and sit back down five times as quickly as possible with his/her arms folded over his/her chest and without using them from a chair of ordinary height (approximately 46 cm seat height). If the patient is not capable of doing this, the number of successful completions are counted rather than the seconds. If the patient takes longer than 10-11 seconds, it must be assumed that he/she is at increased risk of falling.

Table 4 shows the results of the chair-rising test carried out in three osteoporosis patients before beginning therapy with 60 mg of anti-RANKL antibody/denosumab (Prolia)+500 mg of calcium+1000 IE of vitamin D3 (left column) and repeated 6 months later (center column). The improvement is given in the right column in %.

TABLE 4

| Chair-Rising | Chair Rising | Improvement |
|---|---|---|
| 9 sec | 8 sec | +11% |
| 10 sec | 8 sec | +20% |
| 14 sec | 10 sec | +29% |

Therapy reduced the risk of falling by 11-29%.

### Example 4

The following data from an oncological patient (prostate cancer) in Table 5 show the serum levels of calcium and vitamin D3 in administration of 120 mg of denosumab (anti-RANKL antibody, Xgeva) every 4 weeks and the oral administration of 500 mg of calcium daily and 1000 IU of vitamin D3 daily.

In Table 5, the patient received 120 mg of denosumab every 4 weeks and 500 mg of calcium and 1000 IU of vitamin D3:

TABLE 5

| | | 25(OH) | | | 25(OH) | |
|---|---|---|---|---|---|---|
| | | vitamin D3 | | | vitamin D3 | |
| | | serum | | | serum | Calcium |
| | Calcium | concentration; | After | Calcium | concentration; | in diet, |
| Patient | mmol/l | μg/l | weeks | mmol/l | μg/l | mg/day |
| 1 | 2.01 | 36.7 | 13/26 | 2.11/2.12 | 28.3/33.5 | 883 |

The following data from osteoporosis patients in Table 6 show the serum values of calcium and vitamin D3 in administration of 60 mg of anti-RANKL antibody/denosumab (Prolia) every 6 months and the oral administration of 500 mg of calcium daily and 1000 IU of vitamin D3 daily.

In Table 6, the osteoporosis patients received 60 mg of denosumab every 6 months, 500 mg of calcium daily, and 1000 IU of vitamin D3, also daily:

TABLE 6

| | | 25(OH) | | | 25(OH) | |
|---|---|---|---|---|---|---|
| | | vitamin D3 | | | vitamin D3 | |
| | | serum | | | serum | Calcium |
| | Calcium | concentration; | After | Calcium | concentration; | in diet, |
| Patient | mmol/l | μg/l | weeks | mmol/l | μg/l | mg/day |
| 2 | 2.32 | 40.8 | 13/26 | 2.37/2.45 | 50.8/44.1 | 904 |
| 3 | 2.21 | 41.9 | 26 | 2.32 | 31.5 | 1300 |
| 4 | 2.33 | 50 | 13/26 | 2.34/2.32 | 58.1/31.5 | 1136 |
| 5 | 2.31 | 29.2 | 13 | 2.32 | 24.8 | 1372 |
| 6 | 2.39 | 36 | 13 | 2.3 | 29.6 | 819 |

At a 25(OH) vitamin D serum concentration of <20 ng/ml, a severe vitamin D deficiency is considered to be present, which according to the DVO guideline means an increased fracture risk. The data in Example 4 shows that the combination according to the present invention is suitable for normalizing the 25(OH) vitamin D level within 3-6 months to above 20 ng/ml or 20 μg/l and thus for reducing the fracture risk.

As a rule, the calcium serum concentration is in the normal range in patients with osteoporosis. In oncological patients, the calcium concentration in the blood can increase (=tumor-induced hypercalcemia) or decrease due to therapy (therapy-induced hypocalcemia). Patients with a calcium-poor diet are therefore at increased risk for hypocalcemia. Daily supplementation according to the invention with 500 mg of calcium provides an even calcium balance.

An increased PTH level (=secondary hyperparathyroidism) and hypocalcemia induced by anti-resorptive therapy with anti-RANKL antibodies (denosumab)—are risk factors for the occurrence of necrosis of the jaw. For this reason, supplementation according to the present invention with calcium and vitamin D reduces the risk of necrosis of the jaw.

The data show that the combination according to the present invention is suitable for normalizing the 25(OH) vitamin D serum concentration within 3-6 months above 20 ng/ml or 20 μg/l and for achieving a positive calcium balance in almost all the patients. The patients were free of adverse effects, and in particular there was no osteonecrosis of the jaw, non-healing wounds leading to disintegration of the jaw, therapy-induced hypocalcemia, cardiovascular adverse effects such as heart attack, atrial fibrillation, cardiac arrhythmias, secondary hyperparathyroidism, cramps and/or numbness.

Moreover, there were no skeletal-related complications, in particular due to solid tumors, particularly breast, prostate, lung, bowel, or bone cancer (osteosarcoma), no (pathological) fractures, irradiation of the bone, spinal cord compression or bone surgery, no bone metastases, pain in bone metastases, nerve entrapment, deformations due to one or multiple solid tumors such as e.g., breast cancer, prostate cancer, lung cancer, or multiple myeloma.

The present invention is not limited to embodiments described herein; reference should be had to the appended claims.

What is claimed is:

1. A method of administering a pharmaceutical composition comprising anti-RANKL antibodies, calcium and vitamin D to a patient in need thereof, the method comprising:
subcutaneously administering the anti-RANKL antibodies at a dose of 60-180 mg in 1-3 ml of solution every four weeks;
orally administering the calcium at a dose of 400 mg daily; and
orally administering the vitamin D at a dose of 800-1200 IU daily,
wherein,
the patient in need thereof has skeletal-related complications due to solid tumors such as breast, prostate, lung, bowel, or bone cancer (osteosarcoma), pathological fractures, irradiation of the bone, spinal cord compression or bone surgery, bone metastases, pain in bone metastases, nerve entrapment, deformations due to one or more solid tumors such as breast cancer, prostate cancer, lung cancer, or multiple myeloma, and the administering of the pharmaceutical composition comprising anti-RANKL antibodies, calcium and vitamin D to the patient in need thereof at least one of treats and prevents hypocalcemia induced by anti-RANKL antibody therapy, and
the calcium and the vitamin D are provided as a combined single daily dose in a form of effervescent granules, swallowable tablets, swallowable capsules, chewable tablets, or ready-to-use granules.

2. The method as recited in claim 1, wherein,
the anti-RANKL antibody comprises a heavy chain and a light chain,
the heavy chain comprises an amino acid sequence as recited in SEQ ID NO: 2 or in SEQ ID NO: 8, and
the light chain comprises an amino acid sequence as recited in SEQ ID NO: 4 or in SEQ ID NO: 9.

3. The method as recited in claim 1, wherein the anti-RANKL antibody comprises at least one of,
a variable region of a heavy chain of an amino acid sequence as recited in SEQ ID NO: 5, and
a variable region of a light chain of an amino acid sequence as recited in SEQ ID NO: 6.

4. The method as recited in claim 1, wherein the anti-RANKL antibody is a monoclonal antibody, a polyclonal antibody, a monospecific antibody, a bispecific antibody, a bifunctional antibody, a single-chain antibody, a synthetic antibody, a recombinant antibody, a mutated antibody, a human antibody, a humanized antibody, a chimeric antibody, an IgG antibody, an IgA antibody, an IgM antibody, an IgE antibody, an antigen-binding fragment, an antibody construct, Fab, Fab/c, Fv, scFv, Fd, dAb, Fab′ or F(ab′)2.

5. The method as recited in claim 4, wherein,
the antibody construct is Fvs (scFv) comprising individual chains or antibody fusion proteins or a fragment thereof, and
the fragment comprises at least one of a variable region of the light chain and a variable region of the heavy chain.

6. The method as recited in claim 1, wherein the calcium comprises at least one of calcium lactate gluconate, calcium gluconate monohydrate, calcium lactate pentahydrate, calcium citrate, calcium citrate tetrahydrate, calcium carbonate, calcium phosphate, calcium dihydrogen phosphate, calcium hydrogen phosphate, calcium hydrogen phosphate dihydrate, calcium acetate, calcium ascorbate, calcium chloride, calcium glucoheptonate, calcium glycerophosphate, and calcium sulfate.

7. The method as recited in claim 1, wherein the vitamin D comprises at least one of vitamin D, vitamin D3, vitamin D2, a derivative of vitamin D, a derivative of vitamin D3, and a derivative of vitamin D2.

8. The method as recited in claim 1, wherein the vitamin D comprises at least one of calcitriol (1,25-dihydroxyvitamin D3), 1α,25-dihydroxycholecalciferol, alfacalcidol (1α-hydroxyvitamin D3), 24,25-dihydroxyvitamin D3, calcifediol, vitamin D2 and ergocalciferol.

9. The method as recited in claim 1, wherein the pharmaceutical composition further comprises at least one of:
pharmaceutically suitable excipients, liquids and solvents.

10. The method as recited in claim 9, wherein the excipients include lactose, starch, acidifying agents, separating agents, fatty acids, natural and nature-identical and other flavorings and flavoring substances, sweeteners, dyes, silicon dioxide, phenylalanine, gum arabic, saccharose, gelatins, cornstarch, soy oil, glycerol, DL-alpha tocopherol, isomalt, sodium hydrogen carbonate, sodium dihydrogen carbonate, sodium citrate, sodium dihydrogen citrate, carmellose sodium, acesulfame potassium, sodium ascorbate, and triglycerides.

11. The method as recited in claim 10, wherein the excipients are provided dispersed in a pharmaceutically compatible liquid.

12. The method as recited in claim 10, wherein,
the acidifying agents include citric acid, malic acid, acid regulators, sodium hydrogen carbonate, sodium carbonate, humectants, sorbitol, xylitol and inulin,
the separating agents include tricalcium phosphate,
the fatty acids include magnesium salts, and magnesium stearate,
the natural and nature-identical and other flavorings and flavoring substances, sweeteners include sodium cyclamate, aspartame, sodium saccharin, and maltodextrin,
the dyes include red beet juice powder and riboflavin-5′-phosphate,
the silicon dioxide include highly disperse silicon dioxide hydrate,
the triglycerides include medium-chain triglycerides, and
the pharmaceutically compatible liquids include water, isotonic saline solution, isotonic sorbitol-sodium acetate-polysorbate solution, isotonic sorbitol-sodium acetate solution, isotonic glucose solution, and pharmaceutically comparable liquids.

13. The method as recited in claim 9, wherein the solvents or the liquids include water, isotonic saline, or glucose solution.

14. The method as recited in claim 1, wherein,
the pharmaceutical composition comprising the anti-RANKL antibodies is provided as an infusion, an infusion solution concentrate, or as an instant infusion prefilled syringe, and
at least one of the pharmaceutical composition comprising the calcium and the vitamin D is provided in solid or liquid form as an effervescent tablet, a swallowable tablet, a swallowable capsule, a chewable tablet, effervescent granules, ready-to-use granules, a drinking solution, drops, a sublingual spray, either individually or in combination.

15. The method as recited in claim 1, wherein the combined single daily dose of the calcium and the vitamin D is provided in the form of ready-to-use-granules.

16. The method as recited in claim 1, wherein the anti-RANKL antibodies are subcutaneously administered at a dose of 180 mg in 1-3 ml of solution every four weeks.


SEQUENCE LISTING (9 sequences)

SEQ ID NO: 1 - DNA, 1426 bp, Mus musculus
  source 1..1426: mol_type = unassigned DNA
aagcttgaccaccatggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattactgggagtggtggtagtacatactacgcagactccgtgaagggccggttcaccatctccagagacaattccaagaacacgctgtatctgcaaatgaacagcctgagagccgaggacacggccgtatattactgtgcgaaagatccagggactacggtgattatgagttggttcgacccctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaacagcctctccctgtctccgggtaaatgataagtcgac

SEQ ID NO: 2 - protein, 467 aa, Mus musculus
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVSGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 728 bp, Mus musculus
  source 1..728: mol_type = unassigned DNA
tctagaccaccatggaaaccccagcgcagcttctcttcctcctgctactctggctcccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgataagtcgac

SEQ ID NO: 4 - protein, 235 aa, Mus musculus
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFQPREAKVQWKVDNALQSGNSQESVTEQDSKDSTYSLSSTLTLSKADYEKHKVYACEVTHQGLSSPVTKSFNRGEC

SEQ ID NO: 5 - protein, 122 aa, Mus musculus
EVQLLESGGGLVQPGGSLRLSCAASGFTFSSYAMSWVRQAPGKGLEWVSGITGSGGSTYYADSVKGRFTISRDNSKNTLYLQMSSLRAEDTAVYYCAKDPGTTVIMSWFDPWGQGTLVTVSS

SEQ ID NO: 6 - protein, 108 aa, Mus musculus
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 7 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 8 - protein, 452 aa, Homo sapiens
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 215 aa, Homo sapiens
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC